(19) United States
(12) Patent Application Publication
(10) Pub. No.: US 2020/0281948 A1
(43) Pub. Date: Sep. 10, 2020

(54) METHODS FOR TREATING AND PREVENTING C. DIFFICILE INFECTION

(71) Applicant: Paratek Pharmaceuticals, Inc., Boston, MA (US)

(72) Inventors: S. Ken Tanaka, Bellevue, WA (US); Michael P. Draper, Windham, NH (US)

(21) Appl. No.: 16/705,379

(22) Filed: Dec. 6, 2019

Related U.S. Application Data
(63) Continuation of application No. 15/468,354, filed on Mar. 24, 2017, now abandoned.
(60) Provisional application No. 62/320,053, filed on Apr. 8, 2016.
(60) Provisional application No. 62/312,996, filed on Mar. 24, 2016.

(51) Int. Cl. A61K 31/65; A61K 31/4709; A61P 31/04; A61K 9/00; A61K 31/4164; A61K 38/14; A61K 45/06

(52) U.S. Cl. CPC A61K 31/65; A61K 31/4709; A61P 31/04; A61K 9/0019; Y02A 50/473; A61K 38/14; A61K 45/06; Y02A 50/481; A61K 31/4164

(57) ABSTRACT

Methods of treating or preventing a C. difficile infection and the associated pathological conditions related to C. difficile infection, are disclosed.

### RELATED APPLICATIONS

This application is a continuation of U.S. patent application Ser. No. 15/468,354, filed on Mar. 24, 2017; which claims the benefit of U.S. Provisional Application Ser. No. 62/312,996, filed on Mar. 24, 2016 and U.S. Provisional Application Ser. No. 62/320,053, filed on Apr. 8, 2016. The entire contents of each of the foregoing applications are incorporated herein by reference.

### BACKGROUND OF THE INVENTION

Clostridium difficile (C. difficile) is a Gram-positive spore forming bacterial species that is ubiquitous in nature and is especially prevalent in soil. Pathogenic C. difficile strains produce multiple toxins, the most well-characterized of which are enterotoxin (C. difficile toxin A) and cytotoxin (C. difficile toxin B), both of which may produce diarrhea and inflammation in infected patients. Toxins A and B are glucosyltransferases that target and inactivate the Rho family of GTPases. Toxin B (cytotoxin) induces actin depolymerization by a mechanism correlated with a decrease in the ADP-ribosylation of the low molecular mass GTP-binding Rho proteins. Another toxin, binary toxin, also has been previously described, but its role in causing pathological conditions associated with C. difficile infections is not fully understood.

C. difficile is transmitted from person to person by the fecal-oral route. However, the organism forms heat-resistant spores that are not killed by alcohol-based hand cleansers or routine surface cleaning. Thus, these spores survive in clinical environments for long periods. Because of this, the bacteria may be cultured from almost any surface. Once spores are ingested, their acid-resistance allows them to pass through the stomach unscathed. They germinate and multiply into vegetative cells in the colon upon exposure to bile acids.

Infection of the gut with C. difficile is thought to occur when this bacterium replaces normal gut flora that has been compromised, usually following antibiotic treatment for an unrelated infection. The disturbance of normal healthy bacteria may provide C. difficile an opportunity to overrun the intestinal microbiome. Thus, C. difficile associated diarrhea (CDAD) is a type of antibiotic-associated diarrhea, and often, mild cases of CDAD may be treated by discontinuing the offending antibiotics. Ironically, more serious cases require targeted antibiotic treatment, such as treatment with vancomycin or metronidazole, and relapses of CDAD have been reported in up to 20% of cases.

Infection with C. difficile can result in pseudomembranous colitis, or inflammation of the intestines, and in infectious diarrhea CDAD, which is the most frequent cause of mortality associated with gastroenteritis in the healthcare system (Johanesen et al., Genes (Basel), 6, 1347-60, 2015; Cohen et al., Infect. Control Hosp. Epidemiol., 31(5), 431-55, 2010). A recent surveillance study reported an estimated 450,000 infections and 29,000 deaths resulting from C. difficile infection in the United States in 2011 (Lessa et al., N. Engl. J. Med. 372(9):825-34, 2015). Annual costs associated with C. difficile infection were estimated to be about $4.8 billion (Lessa et al., N. Engl. J. Med. 372(9):825-34, 2015).

Antibiotic treatment of C. diffcile infections may be difficult, due both to antibiotic resistance and physiological factors of the bacteria (spore formation and protective effects of the pseudomembrane). The emergence of a new, highly toxic strain of C. difficile, resistant to fluoroquinolone antibiotics, such as ciprofloxacin and levofloxacin, said to be causing geographically dispersed outbreaks in North America, was reported in 2005. The U.S. Centers for Disease Control (CDC) in Atlanta warned of the emergence of an epidemic strain with increased virulence, antibiotic resistance, or both.

Therefore, more effective methods for treating and preventing C. difficile infections and CDAD are needed.

### SUMMARY OF THE INVENTION

Omadacycline, also referred to as Compound A, is a first in class aminomethylcycline having a structure as shown below (Honeyman et al., Antimicrob. Agents Chemother. 59(11), 7044-53, 2015):

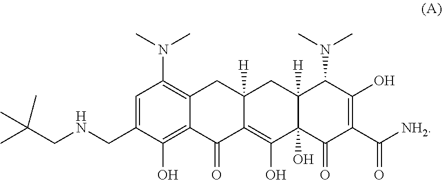 [SMILES: CN(C)c1cc(CNCC(C)(C)C)c(O)c2c1C[C@H]1C[C@H]3[C@H](N(C)C)C(O)=C(C(N)=O)C(=O)[C@@]3(O)C(O)=C1C2=O]

It has been surprisingly discovered that omadacycline exhibits unusually high activity against C. difficile. It has also been surprisingly observed that, unlike other antibiotics, omadacycline is not associated with an increased risk of developing a C. difficile infection.

Accordingly, in some embodiments, the present invention pertains, at least in part, to a method of treating C. difficile infection in a subject in need thereof, the method comprising administering to the subject an effective amount of a compound, or a salt thereof, wherein the compound is compound A′ having the following structural formula:

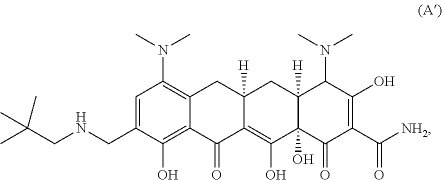 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

such that the C. difficile infection in the subject is treated.

In some embodiments, the C. difficile infection is a recurrent C. difficile infection. In some embodiments, the compound is administered in combination with at least one or more additional therapy used for treating C. difficile infection. In one embodiment, the therapy comprises administering an antibiotic, e.g., metronidazole or vancomycin. In another embodiment, the therapy comprises administering a probiotic. In yet another embodiment, the therapy comprises administering a fecal transplant.

In some embodiments, the present invention also provides a method of treating a bacterial infection without causing C. difficile infection in a subject who is at risk of developing a C. difficile infection, the method comprising administering to the subject an effective amount of a compound, or a salt thereof, wherein the compound is compound A′ having the following structural formula:

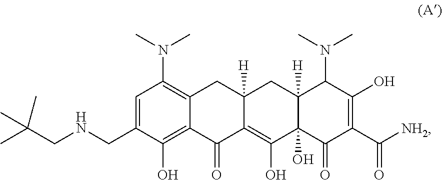 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

such that the bacterial infection in the subject is treated without causing C. difficile infection.

In some embodiments, the present invention also provides a method of treating a bacterial infection without substantially disrupting gut microbiome in a subject who is at risk of developing a C. difficile infection, the method comprising administering to the subject an effective amount of a compound, or a salt thereof, wherein the compound is compound A′ having the following structural formula:

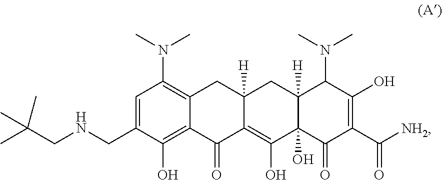 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

such that the bacterial infection in the subject is treated without substantially disrupting gut microbiome.

In certain aspects, treating bacterial infection without substantially disrupting gut microbiome does not result in a C. difficile infection in the subject. In some aspects, the methods of the invention further comprise, prior to administering, selecting a subject at risk of developing a C. difficile infection.

In some embodiments, the present invention provides a method of treating a bacterial infection in a subject who is predisposed to developing a C. difficile infection, the method comprising administering to the subject an effective amount of a compound, or a salt thereof, wherein the compound is compound A′ having the following structural formula:

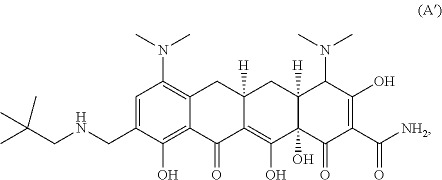 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

such that the bacterial infection in the subject is treated.

In certain aspects, the present invention also provides a method of treating a bacterial infection in a subject who is at risk of developing C. difficile infection, the method comprising the steps of:

selecting a subject at risk of developing a C. difficile infection; and

administering to the subject an effective amount of a compound, wherein the compound is compound A′, or a salt thereof, having the following structural formula:

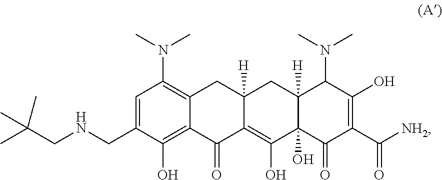 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

such that the bacterial infection in the subject is treated.

In some aspects, the bacterial infection is selected from the group consisting of skin or skin structure infection, community-acquired bacterial pneumonia (CABP) and urinary tract infection (UTI).

In some aspects, the bacterial infection is caused by a gram positive bacterium (e.g., a gram-positive anaerobe). In other aspects, the bacterial infection is caused by a gram negative bacterium (e.g., a gram-negative rod (GNR)). In a further embodiment, the bacterial infection is caused by a bacterium belonging to the species selected from the group consisting of: E. coli, S. aureus, E. faecalis, K. pneumoniae, E. hirae, A. baumanii, B. catarrhalis, H. influenza, P. aeruginosa, and E. faecium.

In a further aspect, the S. aureus is methicillin-susceptible S. aureus (MSSA) or methicillin-resistant S. aureus (MRSA), including both hospital associated and community-associated MRSA. In one embodiment, the infection is a hospital-associated MRSA infection. In another embodiment, the infection is a community-associated MRSA infection.

In one aspect, the bacterial infection is caused by streptococci (e.g., Streptococcus pneumoniae, penicillin-resistant Streptococcus pneumoniae (PRSP), Streptococcus pyogenes, and Streptococcus agalactiae), Viridans Streptococci, Enterococcus, or combinations thereof.

In yet another aspect, the bacterial infection is caused by a bacterium belonging to the genus selected from the group consisting of: Salmonella and Streptococcus.

In an embodiment, the bacterial infection may be resistant to other antibiotics, such as penicillin or tetracycline.

In some embodiments, the compound used in the methods of the invention is Compound A having the following structural formula:

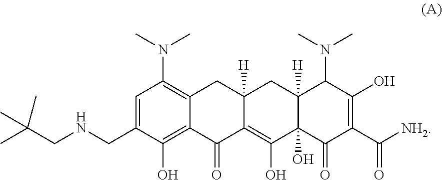 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

In certain aspects, the subject at risk of developing C. difficile infection is a subject who was recently treated with one or more antibiotic, e.g., a broad spectrum antibiotic. In one aspect, the subject at risk of developing C. difficile infection is a subject who has had surgery of the gastrointestinal tract. In another aspect, the subject at risk of developing C. difficile infection is a subject who has a disease of the colon, e.g., an inflammatory bowel disease or colorectal cancer. In one aspect, the subject at risk of developing C. difficile infection is a subject who has a weakened immune system. In another aspect, the subject at risk of developing C. difficile infection is a subject who is on chemotherapy. In yet another aspect, the subject at risk of developing C. difficile infection is a subject who previously had a C. difficile infection. In yet another aspect, the subject at risk of developing C. difficile infection is a subject who is of an advanced age, e.g., 65 years or older. In yet another aspect, the subject at risk of developing C. difficile infection is a subject who has a kidney disease. In one embodiment, the subject at risk of developing C. difficile infection is a subject who takes proton-pump inhibitors.

In one embodiment, the subject at risk of developing C. difficile infection is a subject who is living in an environment that predisposes the subject to developing a C. difficile infection. In a further aspect, the environment that predisposes the subject to developing a C. difficile infection comprises a hospital, a nursing home or an assisted living facility.

In one embodiment, the compound is administered orally. In another embodiment, the compound is administered intravenously. In a further embodiment, the compound is administered as at least one intravenous dose, followed by at least one oral dose. In a further aspect, the at least one oral dose is administered about 24 hours after the at least one intravenous dose.

In one embodiment, the compound is administered once per day or twice per day.

In some embodiments, compound is administered at the dose of about 100 mg, about 200 mg, about 300 mg, about 600 mg or about 900 mg.

In some embodiments, the subject is treated up to and including about 14 days, up to and including about 10 days, up to and including about 9 days, up to and including about 8 days, or up to and including about 7 days.

In one aspect, the pharmaceutically acceptable salt of the compound of the invention is a hydrochloride salt. In another aspect, the pharmaceutically acceptable salt of the compound of the invention is a tosylate salt.

### BRIEF DESCRIPTION OF THE DRAWINGS

FIG. 1 is a Kaplan-Meier plot of percent survival of hamsters infected with C. difficile after treatment with omadacycline and comparators.

FIG. 2 is a schematic showing experimental time frame for the gut model experiment described in Example 3.

FIG. 3 is a graph showing gut microflora populations (log₁₀ cfu/mL) in Vessel 2 of the omadacycine exposed gut model described in Example 3. Periods A-D are as shown in FIG. 2.

FIG. 4 is a graph showing gut microflora populations (log₁₀ cfu/mL) in Vessel 3 of the omadacycine exposed gut model described in Example 3. Periods A-D are as shown in FIG. 2.

FIG. 5 is a graph showing C. difficile counts (log₁₀ cfu/mL), toxin titer (Relative Units RU) and active omadacycline (OMA) concentration (mg/L) in vessel 1 of the omadacycline exposed model described in Example 3. Periods A-D are defined in FIG. 2.

FIG. 6 is a graph showing C. difficile counts (log₁₀ cfu/mL), toxin titer (Relative Units RU) and active omadacycline (OMA) concentration (mg/L) in vessel 2 of the omadacycline exposed model described in Example 3. Periods A-D are defined in FIG. 2.

FIG. 7 is a graph showing C. difficile counts (log₁₀ cfu/mL), toxin titer (Relative Units RU) and active omadacycline (OMA) concentration (mg/L) in vessel 3 of the omadacycline exposed model described in Example 3. Periods A-D are defined in FIG. 2.

FIG. 8 is a graph showing gut microflora populations (log₁₀ cfu/mL) in Vessel 2 of the omadacycine exposed gut model described in Example 4. Periods A-D are as defined in FIG. 2. The horizontal dotted line indicates the limit of detection for viable counts.

FIG. 9 is a graph showing gut microflora populations (log₁₀ cfu/mL) in Vessel 3 of the omadacycine exposed gut model described in Example 4. Periods A-D are defined in FIG. 2. The horizontal dotted line indicates the limit of detection for viable counts.

FIG. 10 is a graph showing C. difficile counts (log₁₀ cfu/mL), toxin titre (Relative Units RU) and active omadacycline (OMA) concentration (mg/L) in vessel 1 of the omadacycline exposed model described in Example 4. Periods A-D are defined in FIG. 2. The horizontal dotted line indicates the limit of detection for viable counts.

FIG. 11 is a graph showing C. difficile counts (log₁₀ cfu/mL), toxin titre (Relative Units RU) and active omadacycline (OMA) concentration (mg/L) in vessel 2 of the omadacycline exposed model described in Example 4. Periods A-D are defined in FIG. 2. The horizontal dotted line indicates the limit of detection for viable counts.

FIG. 12 is a graph showing C. difficile counts (log₁₀ cfu/mL), toxin titre (Relative Units RU) and active omadacycline (OMA) concentration (mg/L) in vessel 3 of the omadacycline exposed model described in Example 4. Periods A-D are defined in FIG. 2. The horizontal dotted line indicates the limit of detection for viable counts.

FIG. 13 is a graph showing gut microflora populations (log₁₀ cfu/mL) in Vessel 2 of the moxifloxacin exposed gut model described in Example 4. Periods A-D are defined in FIG. 2. The horizontal dotted line indicates the limit of detection for viable counts.

FIG. 14 is a graph showing gut microflora populations (log₁₀ cfu/mL) in Vessel 3 of the moxifloxacin exposed gut model described in Example 4. Periods A-D are defined in FIG. 2. The horizontal dotted line indicates the limit of detection for viable counts.

FIG. 15 is a graph showing C. difficile counts (log₁₀ cfu/mL), toxin titre (Relative Units RU) in vessel 1 of the moxifloxacin exposed model described in Example 4. Periods A-D are defined in FIG. 2. The horizontal dotted line indicates the limit of detection for viable counts.

FIG. 16 is a graph showing C. difficile counts (log₁₀ cfu/mL), toxin titre (Relative Units RU) in vessel 2 of the moxifloxacin exposed model described in Example 4. Periods A-D are defined in FIG. 2. The horizontal dotted line indicates the limit of detection for viable counts.

FIG. 17 is a graph showing C. difficile counts (log₁₀ cfu/mL), toxin titre (Relative Units RU) in vessel 3 of the moxifloxacin exposed model in Example 4. Periods A-D are defined in FIG. 2. The horizontal dotted line indicates the limit of detection for viable counts.

### DETAILED DESCRIPTION OF THE INVENTION

### Treatment of C. Difficile Infection

The present invention is based on a surprising discovery that omadacycine exhibits unexpectedly high activity against C. difficile. Accordingly, in some embodiments, the present invention pertains, at least in part, to a method of treating C. difficile infection in a subject in need thereof, e.g, a human subject, the method comprising administering to the subject an effective amount of a compound, or a salt thereof, wherein the compound is compound A′ having the following structural formula:

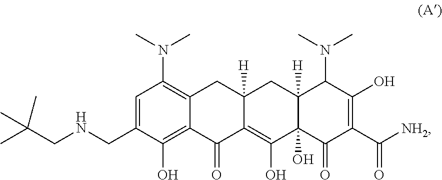 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

such that the C. difficile infection in the subject is treated.

In some embodiments, the compound used in the methods of the invention is Compound A having the following structural formula:

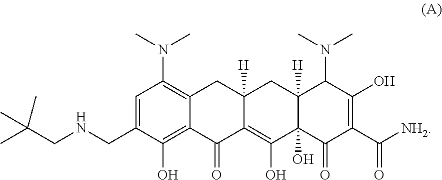 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

In some embodiments, the C. difficile infection may be a recurrent C. difficile infection. Recurrence of C. difficile infection may occur in 20-30% of subjects after treatment of the initial Clostridium difficile infection (CDI) with either metronidazole or vancomycin. Such recurrence is frustrating because there is no approved treatment alternative that provides a lower probability of yet another recurrence. Following a second recurrence, subsequent episodes occur in as many as 40%-60% of subjects. Recurrent CDI may be a consequence of resident spores or infection from local environmental contamination. Relapse and reinfection are therefore difficult to distinguish. Both metronidazole and vancomycin suppress the growth of the normal microflora and thereby defeat natural colonization resistance.

In some embodiments, the C. difficile infection is a superinfection.

In some embodiments, a subject who develops a C. difficile infection, e.g., a recurrent C. difficile infection or a C. difficile superinfection, is a subject who lives in an environment that predisposes a subject to developing a C. difficile infection. Such an environment may comprise any environment in a health care setting, including a hospital, a nursing home or an assisted living facility. An environment in a health care setting may become contaminated with C. difficile spores, and the extent of contamination is proportional to the number of patients with CDAD. Although asymptomatic, colonized patients may also serve as a source of contamination.

The compound of the invention, e.g., Compound (A′) or Compound (A), may be administered in combination with at least one or more additional therapy used for treating C. difficile infection. For example, the therapy may comprise administering an antibiotic that is used for treating C. difficile infection, e.g., metronidazole or vancomycin. The additional therapy may also comprise administering a probiotic, e.g., formulations comprising L. rhamnosus or Saccharomyces boulardii. In yet another embodiment, the additional therapy comprises administering a fecal transplant. Without wishing to be bound by a specific theory, it is believed that administration of a fecal transplant decreases disruptions in intestinal microbiota allowed the C. difficile infection to take hold.

Identification of subjects with C. difficile infection may be done using methods commonly known in the art. Such methods include, but are not limited to, stool culture for C. diffile; molecular tests to detect C. difficile produced toxins A and/or B by, e.g., a PCR-based assay, a tissue culture cytotoxicity assay or an enzyme immunoassay; and detecting the presence of a C. difficile antigen using, e.g., latex agglutination or immunochromatographic assays.

### Treatment of Bacterial Infections

The present invention also provides a method of treating a bacterial infection without causing C. difficile infection in a subject, e.g, a human subject, who is at risk of developing a C. difficile infection, the method comprising administering to the subject an effective amount of a compound, or a salt thereof, wherein the compound is compound A′ having the following structural formula:

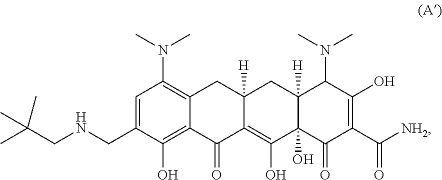 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

such that the bacterial infection in the subject is treated without causing C. difficile infection.

In some embodiments, the compound used in the methods of the invention is Compound A having the following structural formula:

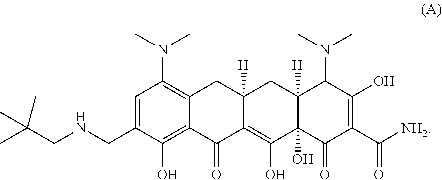 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

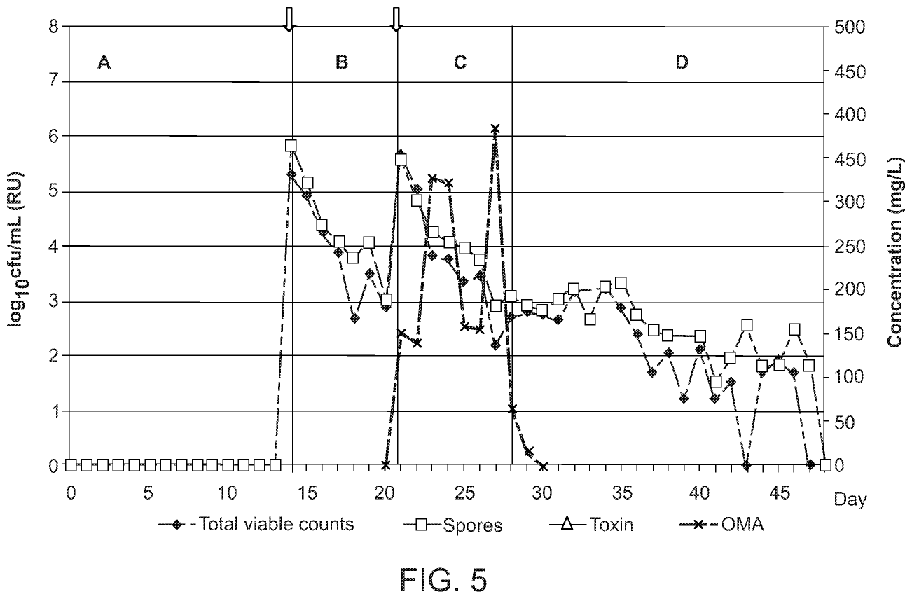

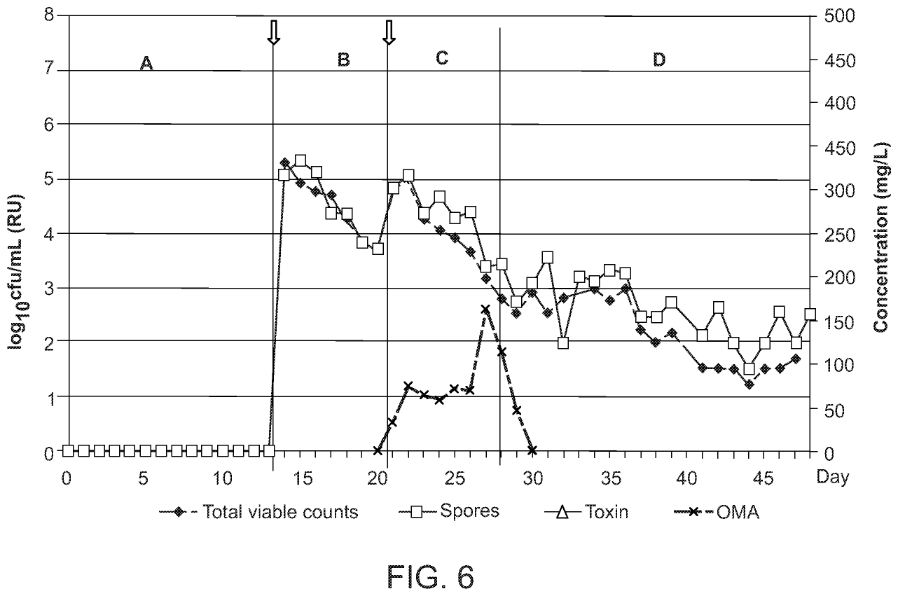

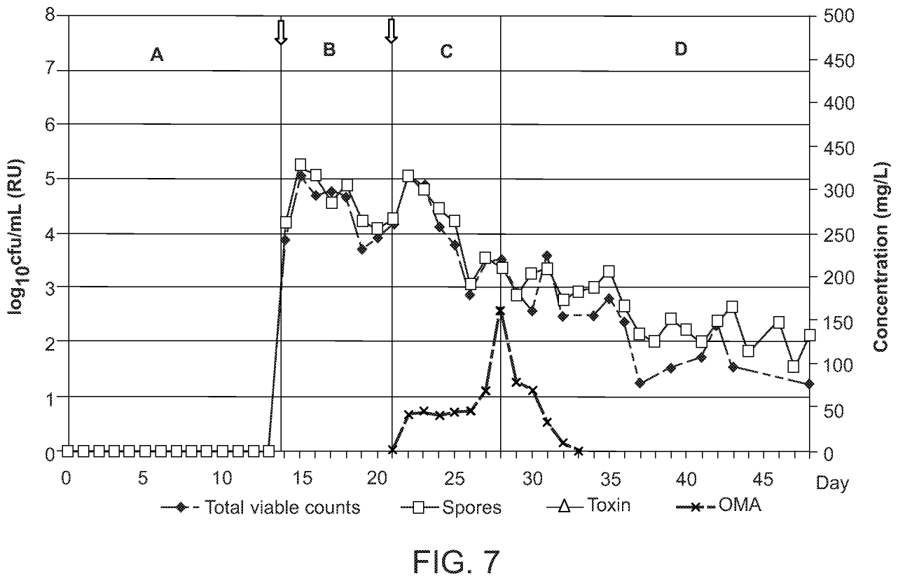

It has been presently discovered that treatment of bacterial infections with omadacycline does not increase the risk of development of a C. difficile infection. This is contrasted with treatment of bacterial infections with other common antibiotics which increases the risk of development of a C. difficile infection and the associated CDAD. Specifically, as described in the ensuing Example 3, an increased omadacycline exposure in an in vitro gut model did not lead to any signs of simulated C. difficile infection. Specifically, C. difficile total viable counts (TVCs) remained roughly equal to spore counts throughout the experiment, indicating that all C. difficile remained as spores, and there was no vegetative cell proliferation observed. In addition, no C. difficile toxin was detected throughout the experiment (see also FIGS. 5, 6 and 7).

In some embodiments, omadacycline exposure or administration of omadacycline to a subject does not promote C. difficile proliferation in vivo.

In some embodiments, omadacycline exposure or administration of omadacycline to a subject has a low potential risk of inducing C. difficile infection.

In some embodiments, the present invention also provides a method of treating a bacterial infection without substantially disrupting gut microbiome in a subject, e.g., a human subject, who is at risk of developing a C. difficile infection, the method comprising administering to the subject an effective amount of a compound, or a salt thereof, wherein the compound is compound A′ having the following structural formula:

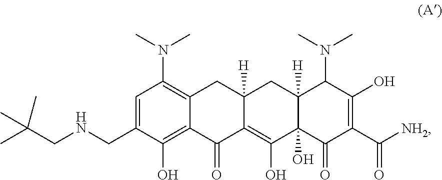 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

such that the bacterial infection in the subject is treated without substantially disrupting gut microbiome.

In some embodiments, the compound used in the methods of the invention is Compound A having the following structural formula:

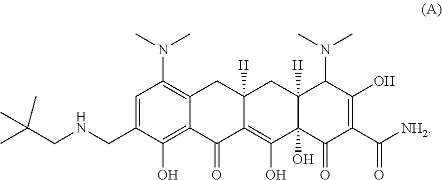 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

The term “without substantially disrupting gut microbiome” refers to levels of modulation of bacterial populations in the gut following treatment with an antibiotic, e.g., omadacycline, such as Compound (A) or Compound (A′), that are not associated with an increased risk of developing a C. difficile infection. This term includes embodiments in which the treatment of a bacterial infection with the compound of the invention, e.g., omadacycline, may result in some disruption of the gut microbiome, but the extent of the disruption does not result in a C. difficile infection or an increased risk of developing a C. difficile infection in the subject. For example, some disruption may occur, but the C. difficile infection is inhibited or prevented by the presence of omadacycline. In at least one embodiment, omadacycline, while extensively disrupting flora or gut microbiome in the gastrointestinal tract, has a low propensity to induce C. difficile infection when administered to a subject.

When an oral dose of omadacycline, e.g., Compound (A) or compound (A′) is administered to a subject, a large proportion of the oral dose, e.g., approximately 60% of the absorbed omadacycline, is eliminated in the gut, i.e., via the biliary/fecal elimination pathway. Because a large proportion of the oral dose of omadacycline is eliminated in the gut, the finding that omadacycline can be administered to a subject without substantially disrupting gut microbiome was surprising and unexpected. Because an infection with C. difficile occurs when gut microbiome is substantially disrupted, the finding that omadacycline, when administered to a subject for treating a bacterial infection, does not increase the subject's risk of developing the C. difficile infection was also surprising and unexpected.

In some embodiments, treating bacterial infection without substantially disrupting gut microbiome does not result in a C. difficile infection in the subject. In some aspects, the methods of the invention further comprise, prior to administering, selecting a subject who is at risk of developing a C. difficile infection or who is predisposed to developing a C. difficile infection.

In some embodiments, the present invention also provides a method of treating a bacterial infection in a subject, e.g., a human subject, who is predisposed to developing a C. difficile infection, the method comprising administering to the subject an effective amount of a compound, or a salt thereof, wherein the compound is compound A′ having the following structural formula:

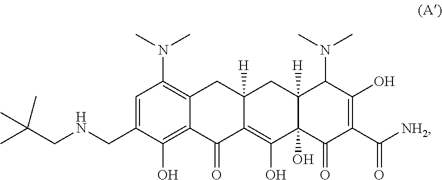 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

such that the bacterial infection in the subject is treated.

In certain aspects, the present invention also provides a method of treating a bacterial infection in a subject who is at risk of developing C. difficile infection, the method comprising the steps of:

selecting a subject at risk of developing a C. difficile infection; and

administering to the subject an effective amount of a compound, wherein the compound is compound A′, or a salt thereof, having the following structural formula:

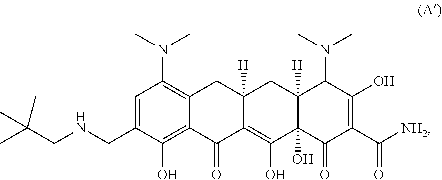 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

such that the bacterial infection in the subject is treated.

In some embodiments, the compound used in the methods of the invention is Compound A having the following structural formula:

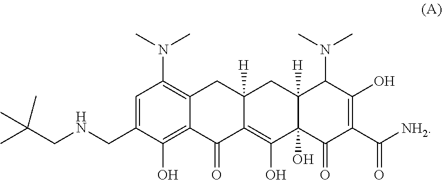 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

The term “a subject who is at risk of developing a C. difficile infection” or “a subject predisposed to developing a C. difficile infection” refers to a subject who is more likely to develop a C. difficile infection as compared to a healthy subject. The term “a subject who is at risk of developing a C. difficile infection” or “a subject predisposed to developing a C. difficile infection” also refers to a subject who lives in an environment that predisposes the subject to developing a C. difficile infection. The factors that may predispose a subject to develop a C. difficile infection may include, but are not limited to, the following:

(a) recent treatment with an antibiotic, e.g., a broad spectrum antibiotic;

(b) having a recent surgical procedure, in particular, a surgical procedure involving a gastrointestinal tract;

(c) having a disease of the colon, e.g., an inflammatory bowel disease or colorectal cancer;

(d) having a weakened immune system, e.g., as a result of a disease or as a result of being treated with chemotherapy;

(e) having previously had at least one a C. difficile infection;

(f) being of an advanced age, e.g., 65 years or older;

(g) having a kidney disease;

(h) taking proton-pump inhibitors; and

(i) living in an environment that predisposes a subject to developing a C. difficile infection. Such environment may comprise any environment in a health care setting, including a hospital, a nursing home or an assisted living facility. An environment in a health care setting may become contaminated with C. difficile spores, and the extent of contamination is proportional to the number of patients with CDAD. Although asymptomatic, colonized patients may also serve as a source of contamination.

Accordingly, in some embodiments, a subject who is at risk of developing a C. difficile infection or a subject who is predisposed to developing a C. difficile infection may belong to at least one of the following categories of subjects:

(a) subjects who had a recent treatment with an antibiotic, e.g., a broad spectrum antibiotic;

(b) subjects who had a recent surgical procedure, in particular, a surgical procedure involving a gastrointestinal tract;

(c) subjects who have a disease of the colon, e.g., an inflammatory bowel disease or colorectal cancer;

(d) subjects who have a weakened immune system, e.g., as a result of a disease or as a result of being treated with chemotherapy;

(e) subjects who previously had at least one a C. difficile infection;

(f) subjects who are of an advanced age, e.g., 65 years or older;

(g) subjects who have a kidney disease;

(h) subjects who are taking proton-pump inhibitors; and

(i) subjects who are living in an environment that predisposes a subject to developing a C. difficile infection. Such an environment may comprise any environment in a health care setting, including a hospital, a nursing home or an assisted living facility. An environment in a health care setting may become contaminated with C. difficile spores, and the extent of contamination is proportional to the number of patients with CDAD. Although asymptomatic, colonized patients may also serve as a source of contamination.

In at least one embodiment, a subject who is at risk of developing a C. difficile infection or a subject who is predisposed to developing a C. difficile infection does not belong to category (f) as listed above, i.e., the subject is not of an advanced age, e.g., 65 years or older.

In some embodiments, a subject who is at risk of developing a C. difficile infection or a subject who is predisposed to developing a C. difficile infection is older than 81 years old. In further embodiments, the subject who is at risk of developing a C. difficile infection or a subject who is predisposed to developing a C. difficile infection is older than 85 years old, older than 90 years old or older than 95 years old.

In some embodiments, the subject who is at risk of developing a C. difficile infection or a subject who is predisposed to developing a C. difficile infection belongs to at least 2, at least 3, at least 4, at least 5, at least 6, at least 7, at least 8 or to all nine categories of subjects as listed above in (a)-(i).

The bacterial infection that may be treated with omadacycline without an increased risk of developing a C. difficile infection may include a skin or skin structure infection (ABSSSI), community-acquired bacterial pneumonia (CABP) and urinary tract infection (UTI).

The bacterial infection may be caused by a gram positive bacterium or a gram negative bacterium. The bacterial infection may be caused by a bacterium belonging to the species selected from the group consisting of: E. coli, S. aureus, e.g., methicillin-resistant S. aureus (MRSA) or methicillin-susceptible S. aureus (MSSA), E. faecalis, K. pneumoniae, E. hirae, A. baumanii, B. catarrhalis, H. influenza, P. aeruginosa, and E. faecium. The bacterial infection may also be caused by a bacterium belonging to the genus selected from the group consisting of: Salmonella and Streptococcus. Treatment of bacterial infections by the compound of the invention, e.g., omadacycline, is described in, e.g., U.S. Pat. Nos. 7,553,828 and 9,265,740, the entire contents of each of which are incorporated herein by reference.

In one embodiment, the compound is administered orally. In another embodiment, the compound is administered intravenously. In a further embodiment, the compound is administered as at least one intravenous dose, followed by at least one oral dose. In a further aspect, the at least one oral dose is administered about 24 hours after the at least one intravenous dose.

In one embodiment, the compound may be administered once per day or twice per day.

The subject may be treated up to and including about 60 days, up to and including 30 days, up to and including 21 days, up to and including 14 days, up to and including about 10 days, up to and including about 9 days, up to and including about 8 days, or up to and including about 7 days.

The pharmaceutically acceptable salt of the compound of the invention may be a hydrochloride salt or a tosylate salt.

### Administration the Compound of the Invention

The compound of the invention, e.g., omadacycline, such as Compound (A′) or Compound (A), or a salt thereof, may be administered as a part of a pharmaceutical composition that comprises, optionally, a pharmaceutically acceptable carrier.

The term “pharmaceutically acceptable carrier” includes substances capable of being co-administered with the compound of the invention, e.g., omadacycline, and which allow the compound of the invention to perform its intended function, e.g., treat or prevent a bacterial infection, e.g., a C. difficile infection. Suitable pharmaceutically acceptable carriers include, but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical compositions can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds of the invention.

The tetracycline compounds of the invention, e.g., omadacycline, are capable of forming a wide variety of salts with various inorganic and organic acids. The acids that may be used to prepare pharmaceutically acceptable acid addition salts of the compound of the invention are those that form nontoxic acid addition salts, i.e., salts containing pharmaceutically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and palmoate [i.e., 1,1′-methylene-bis-(2-hydroxy-3-naphthoate)] salts. Although such salts must be pharmaceutically acceptable for administration to a subject, e.g., a mammal, such as a human, it is often desirable in practice to initially isolate the compound of the invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compound of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. Preferably, the compound of the invention is administered as a tosylate (e.g., p-toluenesulfonate) salt or as a freebase orally or as a hydrochloride salt intravenously.

Salts of the compound of the invention, e.g., omadacycline, are described in, e.g., U.S. Pat. Nos. 8,383,610 and 9,227,921, the entire contents of which are incorporated herein by reference.

In yet another further embodiment, the compound of the invention may be administered at a dose of from about 110 to about 490 mg, from about 120 to about 480 mg, from about 130 to about 470 mg, from about 140 to about 460 mg, from about 150 to about 450 mg, from about 160 to about 440 mg, from about 170 mg to about 430 mg, from about 180 mg to about 420 mg, from about 190 mg to about 410 mg, from about 200 mg to about 400 mg, from about 210 mg to about 390 mg, from about 220 mg to about 380 mg, from about 230 mg to about 370 mg, from about 240 mg to about 360 mg, from about 250 mg to about 350 mg, from about 260 mg to about 340 mg, from about 270 mg to about 330 mg, from about 280 mg to about 320 mg, from about 290 mg to about 310 mg, or about 300 mg of the compound of the invention, e.g., omadacycline.

In some embodiments, a compound of the invention, e.g., Compound A′ or Compound A, may be administered at a dose of from about 10 to about 1000 mg, about 20 to about 750 mg, about 50 to about 500 mg, about 75 to about 400 mg, about 100 to about 300 mg, about 110 to about 290 mg, about 120 to about 280 mg, about 130 to about 270 mg, about 140 to about 260 mg, about 150 to about 250 mg, about 160 to about 240 mg, about 170 mg to about 230 mg, about 180 mg to about 220 mg, about 190 mg to about 210 mg, or about 200 mg. In another embodiment, the compound of the present invention, e.g., Compound A′ or compound A, may be administered intravenously at a dose of about 5 to about 500 mg, about 10 to about 400 mg, about 25 to about 300 mg, about 50 to about 200 mg, about 50 to about 150 mg, about 60 to about 140 mg, about 70 mg to about 130 mg, about 80 mg to about 120 mg, about 90 mg to about 110 mg, or about 100 mg. In one embodiment, the compound of the invention, e.g., Compound A′ or Compound A, may be administered orally at a dose of from about 5 to about 800 mg, about 10 to about 700 mg, about 25 to about 600 mg, about 50 to about 500 mg, about 100 to about 400 mg, about 150 to about 350 mg, about 200 mg to about 340 mg, about 250 mg to about 330 mg, about 270 mg to about 320 mg, about 280 to about 310, or about 300 mg.

In some embodiments, the compound of the invention is administered at a dose of about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg or about 600 mg. In a further embodiment, the dose is an intravenous dose. In another further embodiment, the dose is an oral dose.

It should be understood that dose ranges comprising the above listed doses are also included in the present invention. For example, any of the above doses may be a lower part or an upper part of a dose range that is included in the present invention. Even further, it should be understood that all lists or collections of numerical values used throughout the present application also are intended to include ranges of the numerical values wherein any of the listed numerical values can be the lower part or upper part of a range. These ranges are intended to be included in the present invention.

In an embodiment, the compound of the invention, e.g., Compound A′ or Compound A, may be administered intravenously at the dose of about 100 mg, about 200 mg, or about 300 mg. In another embodiment, the compound of the invention, e.g., Compound A′ or Compound A, may be administered orally at the dose of about 300 mg, about 600 mg, or about 900 mg.

In one embodiment, an oral dose of compound of the invention, e.g., Compound A′ or Compound A is 3 times larger than an intravenous dose of the compound of the invention, e.g., Compound A′ or Compound A.

It will be understood that for all listed embodiments the dose of the compound of the invention, e.g., Compound A′ or Compound A, is also an effective amount of the compound of the invention, e.g., Compound A′ or Compound A.

In one embodiment, the effective amount of a compound of the present invention, e.g., Compound A or Compound A′, when administered orally, is from about 10 to about 1000 mg, about 20 to about 750 mg, about 50 to about 500 mg, about 75 to about 400 mg, about 100 to about 300 mg, about 110 to about 290 mg, about 120 to about 280 mg, about 130 to about 270 mg, about 140 to about 260 mg, about 150 to about 250 mg, about 160 to about 240 mg, about 170 mg to about 230 mg, about 180 mg to about 220 mg, about 190 mg to about 210 mg, or about 200 mg. In another embodiment, the effective amount of a compound of the present invention, e.g., Compound A or compound A′, when administered intravenously, is from about 5 to about 500 mg, about 10 to about 400 mg, about 25 to about 300 mg, about 50 to about 200 mg, about 50 to about 150 mg, about 60 to about 140 mg, about 70 mg to about 130 mg, about 80 mg to about 120 mg, about 90 mg to about 110 mg, or about 100 mg.

The compound of the invention, e.g., omadacycline, and pharmaceutically acceptable salts thereof may be administered via either the oral, parenteral or topical routes. In general, the compound of the invention is most desirably administered in an effective dosage, depending upon the weight and condition of the subject being treated and the particular route of administration chosen. Variations may occur depending upon the species of the subject being treated and its individual response to said medicament, as well as on the type of pharmaceutical formulation chosen and the time period and interval at which such administration is carried out.

The pharmaceutical compositions of the invention may be administered alone or in combination with other known compositions for treating bacterial infections in a subject, e.g., a mammal. Mammals include pets (e.g., cats, dogs, ferrets, etc.), farm animals (cows, sheep, pigs, horses, goats, etc.), lab animals (rats, mice, monkeys, etc.), and primates (chimpanzees, humans, gorillas). The language “in combination with” a known composition is intended to include simultaneous administration of the compound of the invention and the known composition, administration of the compound of the invention first, followed by the known composition and administration of the known composition first, followed by the compound of the invention. Any of the therapeutic compositions known in the art for treating bacterial infections, e.g., a C. difficile infection, may be used in the methods of the invention.

The compound of the invention may be administered alone or in combination with pharmaceutically acceptable carriers or diluents by any of the routes previously mentioned, and the administration may be carried out in single or multiple doses. For example, the compound of the invention may be administered advantageously in a wide variety of different dosage forms, i.e., it may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the compound of this invention is present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols.

When aqueous suspensions and/or elixirs are desired for oral administration, the active ingredient, i.e., omadacycline, may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration (including intraperitoneal, subcutaneous, intravenous, intradermal or intramuscular injection), solutions of the compound of the invention, e.g., omadacyline, in either sesame or peanut oil or in aqueous propylene glycol may be employed. The aqueous solutions should be suitably buffered (e.g., have a pH greater than 8) if necessary and the liquid diluent first rendered isotonic.

These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art. For parenteral application, examples of suitable preparations include solutions, preferably oily or aqueous solutions as well as suspensions, emulsions, or implants, including suppositories. Omadacycline may be formulated in sterile form in multiple or single dose formats such as being dispersed in a fluid carrier such as sterile physiological saline or 5% saline dextrose solutions commonly used with injectables.

For enteral application, particularly suitable are tablets, dragees or capsules having talc and/or carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc.

In addition to treatment of human subjects, the therapeutic methods of the invention also will have significant veterinary applications, e.g. for treatment of livestock such as cattle, sheep, goats, cows, swine and the like; poultry such as chickens, ducks, geese, turkeys and the like; horses; and pets such as dogs and cats.

In some embodiments, a compound of the present invention, e.g., Compound A or Compound A′, may be administered for at least 3 days, at least 7 days, at least 14 days, at least 21 days, at least 30 days or at least 60 days. For example, the administration of the compound of the present invention may last for 3 days to 7 days, for 3 days to 14 days, for 3 days to 21 days, for 3 days to 30 days, for 3 days to 60 days, for 7 days to 14 days, for 7 days to 21 days, for 7 days to 30 days, for 7 days to 60 days, for 14 days to 21 days, for 14 days to 30 days, for 14 days to 60 days, for 21 days to 30 days, for 21 days to 60 days, or for 30 days to 60 days.

For example, a compound of the present invention, e.g., Compound A or Compound A′, may be administered for 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days or 60 days.

In some embodiments, the method comprises administering to the subject one or more loading doses of the compound, followed by one or more maintenance doses of the compound. In one embodiment, the one or more loading dose may be greater than the one or more maintenance dose.

In some embodiments, administration of a compound of the present invention, e.g., Compound A or Compound A′, to a subject may comprise administering one or more loading doses of the compound, followed by one or more maintenance doses of the compound. In some embodiments, the one or more loading dose of the compound may be greater than the one or more maintenance dose of the compound. For example, the loading dose may be about 200 mg, while the maintenance dose may be about 150 mg, 100 mg or 50 mg; or the loading dose may be about 400 mg, while the maintenance dose may be about 300 mg, 250 mg, 200 mg, 150 mg, 100 mg or 50 mg; or the loading dose may be about 100 mg, while the maintenance dose may be about 75 mg, about 50 mg or about 25 mg.

The loading dose of the compound of the invention and the maintenance dose of the compound of the invention may be administered via the same route or different routes. For example, the loading dose(s) may be administered intravenously and the maintenance dose may be administered orally. In other embodiments, both the loading dose(s) and the maintenance doses may be administered orally, or both the loading dose(s) and the maintenance dose may be administered intravenously.

In some embodiments, the loading dose of the compound of the invention, e.g., Compound A′ or Compound A, may be an oral dose or an intravenous dose administered twice daily, and the maintenance dose may be an oral dose or an intravenous dose administered once daily. For example, the compound of the invention, e.g., Compound A′ or Compound A, may be administered as an intravenous loading dose of 100 mg twice daily, followed by an intravenous maintenance dose of 100 mg once daily. In another example, the compound of the invention, e.g., Compound A′ or Compound A, may be administered as an intravenous loading dose of 100 mg twice daily, followed by an oral maintenance dose of 300 mg once daily. In yet another example, the compound of the invention, e.g., Compound A′ or Compound A, may be administered as an oral loading dose of 300 mg twice daily, followed by an oral maintenance dose of 300 mg once daily.

In another embodiment, the compound of the present invention, e.g., Compound A or Compound A′, may be administered once per day or twice per day, either intravenously or orally.

The term “treating” or “treatment” refers to the amelioration or diminishment of one or more symptoms of the disorder, e.g., a bacterial infection, to be treated.

The term “prophylaxis”, “prevent”, or “prevention” means to prevent or reduce the risk of a bacterial infection.

The term “resistance” or “resistant” refers to the antibiotic/organism standards as defined by the Clinical and Laboratories Standards Institute (CLSI) and/or the Food and Drug Administration (FDA).

The term “subject” includes animals which are subject to a bacterial infection. Examples of subjects include animals such as farm animals (e.g., cows, pigs, horses, goats, rabbits, sheep, chickens, etc.), lab animals (mice, rats, monkeys, chimpanzees, etc.), pets (e.g., dogs, cats, ferrets, hamsters, etc.), birds (e.g., chickens, turkeys, ducks, geese, crows, ravens, sparrows, etc.), primates (e.g., monkeys, gorillas, chimpanzees, bonobos, and humans), and other animals (e.g., squirrels, raccoons, mice, rats, etc.). In one embodiment, the subject is a mouse or rat. In one embodiment, the subject is a cow, a pig, or a chicken. In one embodiment, the subject is a human.

The term “effective amount” includes the amount of a compound of the present invention needed to treat or prevent a bacterial infection. For example, an effective amount describes an efficacious level sufficient to achieve the desired therapeutic effect through the killing of bacteria and/or inhibition of bacterial growth. In one embodiment, the effective amount is sufficient to eradicate the bacterium or bacteria causing the infection.

The term “about” refers to a range of values which can be 15%, 10%, 8%, 5%, 3%, 2%, 1%, or 0.5% more or less than the specified value. For example, “about 10%” can be from 8.5% to 11.5%. In one embodiment, the term “about” refers to a range of values which are 5% more or less than the specified value. In another embodiment, the term “about” refers to a range of values which are 2% more or less than the specified value. In another embodiment, the term “about” refers to a range of values which are 1% more or less than the specified value.

The structures of the compound of the present invention includes double bonds or asymmetric carbon atoms. Such compounds can occur as racemates, racemic mixtures, single enantiomers, individual diastereomers, diastereomeric mixtures, and cis- or trans- or E- or Z-double bond isomeric forms. Such isomers can be obtained in substantially pure form by classical separation techniques and by stereochemically controlled synthesis. Furthermore, the structures and other compounds and moieties discussed in the present invention also include all tautomers thereof.

It is to be understood that wherever values and ranges are provided herein, e.g., in ages of subject populations, dosages, and time durations, etc., all values and ranges encompassed by these values and ranges, are meant to be encompassed within the scope of the present invention. Moreover, all values in these values and ranges may also be the upper or lower limits of a range.

The compounds of the present invention may be synthesized and purified according to the synthetic scheme as shown below and as described in US 2008/0287401, the entire contents of which are incorporated herein by reference.

The efficacy of the compound of the present invention in treating or preventing a bacterial infection may be assessed by using common methods known in the art. In one embodiment, the efficacy may be determined by Minimum Inhibition Concentration (MIC) assay. For example, the compound of the present invention may be serially diluted and then added to the growth medium, e.g., cation-adjusted Mueller Hinton broth (CAMHB) of the bacterial culture. The lowest concentration of the compound of the present invention that inhibits 50% or 90% bacterial growth (i.e., MIC₅₀ or MIC₉₀) is determined and, if necessary, compared with MIC₅₀ or MIC₉₀ of other antibiotics. In another embodiment, the efficacy may be determined through in vivo assays known in the art (e.g., animal experiments). For example, the compound of the present invention is administered to experimental animals (e.g., mice and rats) at decreasing amounts. The lowest amount of the compound of the present invention that treats the experimental animal (e.g., ameliorates symptoms of a bacterial infection, prolongs the survival time of the animal, and allows animal to survive the bacterial infection) or prevents the experimental animals from being infected by the bacterium or developing any symptoms of the infection is determined and, if necessary, compared with the lowest amount of other antibiotics which achieves the same results.

### EXEMPLIFICATION OF THE INVENTION

### Example 1

### In Vitro Activity of Omadacycline (Compound A) Against C. difficile Strains

### Materials and Methods

The activity of omadacycline was tested in vitro against 27 clinical isolates of C. difficile. This activity was compared to the activity against C. difficile of other comparator antibiotics that included cefotaxime, doxycycline, amoxicillin clavulanate, metronidazole, imipenem and clindamycin. The experiments were carried out using broth and agar microdilution methods according to Clinical and Laboratory Standards Institute (CLSI) guidelines. Wilkins-Chalgren broth containing each test antibiotic at the final concentration of 0.016 mg/mL to 16 mg/mL was added to the 96-well plates, which were incubated for 48 hours under anaerobic conditions. Each test was run in duplicate.

### Results

The minimum inhibitory concentrations (MIC₅₀ and MIC₉₀) for omadacycline and other antibiotics are shown in Table 1. Specifically, MIC₉₀ for omadacycline against C. difficile was 0.06 mg/L by broth dilution and 0.12 mg/L by agar dilution. Omadacycline was more active than doxycycline (MIC₉₀ of 0.5 mg/L by broth and 1 mg/L by agar dilution).

TABLE 1
Minimum inhibitory concentration for omadacycline and comparator antibiotics
against C. difficile strains (N = 27) by broth and agar dilution methods.

| | Minimum Inhibitory Concentration (mg/mL) | | | | | |
|---|---|---|---|---|---|---|
| | Broth Microdilution | | | Agar Microdilution | | |
| Drug | Range | MIC₅₀ | MIC₉₀ | Range | MIC₅₀ | MIC₉₀ |
| Omadacycline | 0.06-0.12 | 0.06 | 0.06 | 0.06-0.12 | 0.12 | 0.12 |
| Cefotaxime | 4->128 | 64 | 128 | 4->128 | >64 | >128 |
| Doxycycline | 0.015-0.5 | 0.03 | 0.5 | 0.03-2 | 0.03 | 1 |
| Amoxicillin | 0.12-0.5 | 0.25 | 0.5 | 0.25-1 | 0.05 | 1 |
| Clavulanate | | | | | | |
| Metronidazole | 0.12-1 | 0.12 | 0.5 | 0.06-0.5 | 0.12 | 0.25 |
| Imipenem | 0.5-8 | 4 | 8 | 0.5-8 | 4 | 8 |
| Clindamycin | 0.12->16 | 4 | >16 | 0.12->16 | 8 | >16 |

The results shown in Table 1 indicate that omadacycline exhibits potent in vitro activity against C. difficile that is similar to the activity of comparator antibiotics.

### Example 2

### In Vitro and In Vivo Activity of Omadacyline Against C. difficile in a Hamster Model of C. difficile-Associated Diarrhea

### Materials and Methods

The activity of omadacycline was determined in the hamster model of C. difficile-associated diarrhea (ViviSource Laboratories, Inc., Waltham Mass.). Male LGV-Golden Syrian Hamsters (Charles River Laboratories Inc., Wilmington, Mass.) weighing 80-100 g were used. Hamsters were kept in a room maintained at 64-76° F. (17.8-24.4° C.) with humidity set at 40%-70%, and standard rodent diet and water were available ad libitum. Hamsters were pretreated with a subcutaneous (SC) dose of 10 mg/kg clindamycin at 24 hours prior to injection.

C. difficile strain ATCC 43596 was obtained from the American Type Culture Collection, (Manassas, Va.) and cultured from freezer stocks under anaerobic conditions on Brucella agar with 5% sheep blood. Hamsters were infected 24 hours after pretreatment with clidamycin with a suspension of a 48 hour culture of C. difficile ATCC 43596, using the dose of 10 mL/kg administered by oral gavage. This resulted in an inoculum of approximately 1.3×10⁷ CFU/hamster. At 24 hours post infection, groups of animals (N=10) received an oral dose of 50 mg/kg/day of omadacycline; 50 mg/kg/day of vancomycin or vehicle (sterile water) for 5 days. Animals were observed daily to assess general health, and body weight was recorded at least 3 times weekly. In vitro activity also was determined for clindamycin, tigecycline, vancomycin, and metronidazole.

Percent survival for each group was determined for up to 21 days post infection. Kaplan-Meier survival analysis was performed with a staircase plot. P-values, significant difference in curves, and median survival were determined using a Log Rank analysis of data,

### Results

Omadacycline was as active in vitro as tigecycline, metronidazole, and vancomycin (MIC90=0.06 mg/L for all drugs) against the infection model C. difficile strain ATCC 43596, while clindamycin exhibited no activity. The results of the in vitro tests against the infection model strain ATCC 43596 are presented in Table 2.

TABLE 2
Minimum inhibitory concentration (MIC90) for omadacycline
and comparator antibiotics against C. difficile strain
ATCC 43596. Place A and plate B are replicates.

MIC₉₀ (mg/L)

| Compound | Plate A | Plate B |
|---|---|---|
| Omadacycline | 0.06 | 0.06 |
| Tigecycline | 0.06 | 0.06 |
| Metronidazole | 0.06 | 0.06 |
| Clindamycin | >32 | >32 |
| Vancomycin | 0.06 | 0.06 |

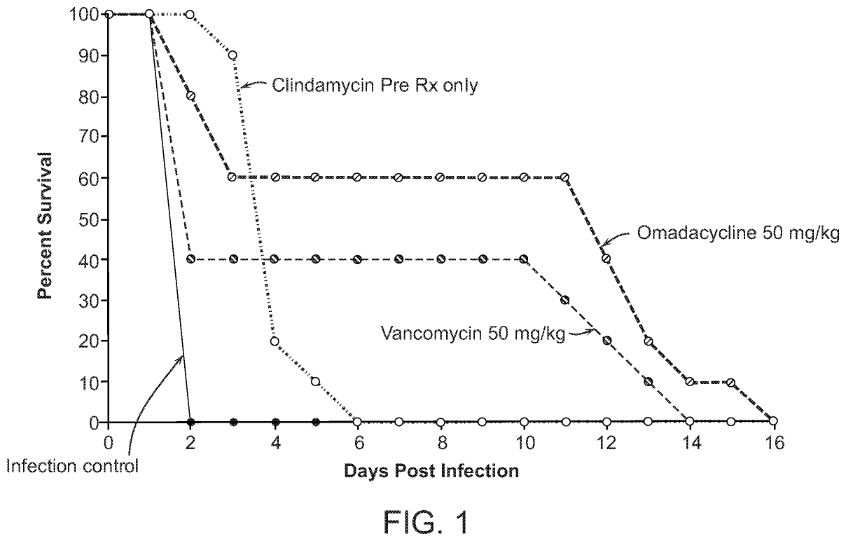

Shown in FIG. 1 is the Kaplan-Meier analysis of percent survival of C. difficile infected hamsters after treatment with omadacycline and comparator antibiotics. Specifically, at day 2 post infection, 100% of omadacycline treated animals were alive, as compared to 40% of animals who received vancomycin and 0% of animals who received vehicle control. Hamsters who received only clindamycin pre-treatment demonstrated 10% of survival at day 2 post infection. For omadacycline treated animals, survival declined to 60% by day 3 and remained at 60% until declining to 40% on day 13, and to 0% on day 16. Animals treated with vancomycin that survived the initial 2 days post infection, exhibited 30% survival by day 11, and all animals succumbed to the infection by day 14. Overall, the median survival for omadacycline treated animals was day 12, as compared to 2 days for vancomycin and 4 days for clindamycin pre-treatment, as shown in Table 3 below.

TABLE 3
Median survival for hamsters after treatment
with omadacycline and comparator antibiotics.

| Test Compound | Median Survival (days) | p-value* |
|---|---|---|
| Omadacycline | 12 | 0.0004 |
| Vancomycin | 2 | 0.0293 |
| Clindamycin | 4 | <0.0001 |

*Kaplan-Meier analysis using log rank test

The data presented in FIG. 1 and in Tables 2 and 3 demonstrate that omadacycline exhibits potent in vitro and in vivo activity against C. difficile in the hamster model of C. difficile-associated diarrhea. In vivo, this activity is superior to the activity of vancomycin.

### Example 3

### Effect of Omadacycline on Gut Microflora and on C. difficile Germination, Proliferation and Toxin Production in an In Vitro Model of Human Gut

### Aims

To determine, using an in vitro model of C. difficile Infection (CDI), the effects of omadacyline instillation on normal gut microflora populations, and to investigate the propensity of omadacycline to induce C. difficile germination, proliferation and toxin production.

### Introduction

An in vitro gut model was used to study the effects of omadacycline instillation on both normal microflora populations and C. difficile. This gut model has been validated against gut contents from sudden death victims and provides a very close simulation of bacterial activities and composition in different areas of the hindgut (Macfarlane et al., Microb. Ecol. 35, 180-7, 1998). The model consists of three vessels aligned in series, and top-fed with a complex growth medium. All three vessels are continuously stirred, anaerobically maintained at 37° C. and regulated to reflect in vivo differences, including pH, from proximal to distal colon. The three anaerobic fermentation vessels are maintained at increasing alkalinity (from pH 5.5±0.2 for vessel 1; pH 6.2±0.2 for vessel 2; and pH 6.8±0.2 for vessel 3). The increasing alkalinity in combination with the nutrient limited conditions are designed to simulate the human gut from the proximal to the distal colon. Inoculation with pooled human feces (from healthy elderly volunteers) is followed by a period of equilibration, during which bacterial populations respond to their environmental conditions and reach a steady state. At this stage, dietary ingredients, prebiotics, pathogens and/or antibiotics may be added, and the bacterial populations monitored. Specific components of the gut flora and relevant pathogens may be closely monitored and their behavior analyzed.

The gut model has been previously used to simulate CDI using epidemic virulent strains (Freeman et al., J. Antimicrob. Chemother. 52, 96-102, 2003). It was shown that cefotaxime, an antibiotic well known for its ability to predispose subjects to CDI, promotes C. difficile germination and toxin production in the gut model. Conversely, piperacillin-tazobactam and tigecycline, antibiotics believed to have a low propensity to induce CDI, do not promote C. difficile germination and toxin production (Baines et al., J. Antimicrob. Chemother. 55, 974-82, 2005; Baines et al., J. Antimicrob. Chemother., 58, 1062-5, 2006). Clindamycin also causes marked toxin production in the gut model, but this can be reversed by dosing the model with a therapeutic agent (Freeman et al., J. Antimicrob. Chemother., 56, 717-25, 2005).

It is believed that the gut model circumvents many of the problems encountered during in vivo studies; including variability of the data derived from fecal specimens, and ethical issues associated with animal testing. Moreover, greater experimental control affords the investigators a level of reproducibility, which would be difficult to achieve in vivo without substantial numbers of subjects/animals. In summary, it is believed that the gut model predictably reflects CDI induction. An understanding of the propensity of novel antimicrobials to induce CDI is of key importance to inform prescription practices.

### Methods

A chemostat gut model was set up as shown in FIG. 2. The gut model was inoculated with pooled fecal slurry (5 volunteers≥60 years of age with no history of antibiotic therapy in the previous 3 months) and left for 2 weeks to allow the bacterial populations to achieve steady state. A single inoculum (˜10⁷ cfu/mL) of C. difficile spores (PCR ribotype 027 strain 210) was added into vessel 1 of the gut model on day 14. One week later, on day 21, a second aliquot of C. difficile spores was added, and antibiotic instillation commenced. Omadacycline instillation (430 mg/L, once daily, for 7 days) commenced on day 21.

Gut microbiota bacterial populations and C. difficile total viable counts and spore counts were enumerated daily by culture on selective and non-selective agars. C. difficile populations were monitored in all three vessels, and all other bacterial groups (total obligate anaerobes, total facultative anaerobes, lactose fermenting enterobacteriaceae, enterococci, total clostridia, lactobacilli, bifidobacteria, B. fragilis group) were monitored in vessels 2 and 3 only. Vessel 3 is of most physiological relevance in terms of propensity to induce CDI. C. difficile total viable counts and spores counts were monitored using viable counting and a differential alcohol shock viable count on selective agars.

From day 14 onwards C. difficile cytotoxin was measured using a quantitative VERO cell cytotoxicity assay. One mL samples were centrifuged at 16,000×g for 15 minutes, and the supernatants were removed. Culture supernatants from the gut model were serially diluted 1:10 in sterile PBS to 10⁻⁶. Twenty microliters of the appropriate dilution was added to vero cell monolayers, and a further 20 μL aliquot of C. sordellii antitoxin (diluted 1:10 in sterile distilled water) was placed in to the corresponding antitoxin row. Monolayers were examined after 24 and 48 hours incubation in 5% CO₂, with a positive result indicated by the presence of cell rounding with concurrent neutralization of effect by C. sordellii antitoxin. Cytotoxin titers (relative units, RU) were an arbitrary log₁₀ scale and the cytotoxin titer reported in the highest dilution with >70% cell rounding, i.e. 10⁰=1RU, 10⁻¹=2RU, 10⁻²=3RU. Samples were taken daily from day 21 onwards to determine antimicrobial concentrations in gut model vessels by bioassay. Concentrations of omadacyline were measured by large-plate bioassay using Wilkins Chalgren agar with Kocuria rhizophila as the indicator organism.

### Results

### Omadacycline Exposed Model

Bioactive omadacycline concentrations peaked at ˜370 mg/L, ˜150 mg/L and ˜150 mg/L in vessels 1, 2 and 3 of the omadacyline exposed model, respectively (FIGS. 5, 6 and 7).

Changes in gut microflora populations were similar in vessels 2 and 3 (FIGS. 3 and 4). Omadacycline instillation caused marked declines in Clostridia (˜6 log₁₀ cfu/mL) and Bifidobacteria (˜6 log₁₀ cfu/mL) populations, which fell below the limit of detection. Decreases in B. fragilis group (˜3 log₁₀ cfu/mL), Lactobacillus spp. (˜2 log₁₀ cfu/mL) and Enterococcus spp. (˜4 log₁₀ cfu/mL) were also observed. Overall, Enterobacteriacea populations remained undisturbed. All populations recovered following the end of omadacyline dosing, and had returned to steady state levels approximately 1 week post antimicrobial exposure.

Despite extensive disruption of gut microflora population, omadacyline exposure did not lead to any signs of simulated C. difficile infection. C. difficile total viable counts (TVCs) remained roughly equal to spore counts throughout the experiment in all three vessels, indicating that all C. difficile remained as spores. There was no vegetative cell proliferation observed. No toxin was detected throughout the experiment in any vessels (FIGS. 5, 6 and 7).

### Discussion

Despite causing extensive disruption to the gut microflora, omadacycine exposure did not induce any signs of simulated CDI within the in vitro human gut model. This model has been shown to be clinically reflective. Antibiotics known to have a high propensity to induce CDI clinically have induced CDI in this model, e.g., clindamycin, cephalosporins and co-amoxyclav, whereas antibiotics described as “low-risk” for CDI clinically have not induced simulated CDI in the gut model, e.g., tigecycline, and piperacillin-tazobactam. See Saxton et al., Antimicrob. Agents and Chemother., 53, 412-420, 2009; Freeman et al., J. Antimicrob. Chemother. 52, 96-102, 2003; Chilton et al., J. Antimicrob. Chemother., 67(4), 951-4, 2012; Baines et al., J. Antimicrob. Chemother., 58, 1062-5, 2006; Baines et al., J. Antimicrob. Chemother., 55, 974-82, 2005. The current data indicates that omadacyline is associated with a low risk for CDI induction, despite the disruptive effect on gut microflora.

### Example 4

### Effect of Omadacycline on Gut Microflora and on C. difficile Germination, Proliferation and Toxin Production in an In Vitro Model of Human Gut

### Aims

To determine, using an in vitro model of C. difficile Infection (CDI), the effects of omadacyline instillation on normal gut microflora populations, and to investigate the propensity of omadacycline to induce C. difficile germination, proliferation and toxin production.

### Methods

A chemostat gut model was set up as shown in FIG. 2. The gut model was inoculated with pooled fecal slurry (5 volunteers≥60 years of age with no history of antibiotic therapy in the previous 3 months) and left for 2 weeks to allow the bacterial populations to achieve steady state. A single inoculum (˜10⁷ cfu/mL) of C. difficile spores (PCR ribotype 027 strain 210) was added into vessel 1 of the gut model on day 14. One week later, on day 21, a second aliquot of C. difficile spores was added, and antibiotic instillation commenced. Model A (LHS) was exposed to moxifloxacin (43 mg/L, once daily, for 7 days) and Model B (RHS) was exposed to omadacycline (430 mg/L, once daily, for 7 days) commenced on day 21.

Bacterial populations in the gut model were monitored using selective agars to count viable bacterial colonies. Populations were monitored every other day for the first 2 weeks until the steady state was reached, and daily thereafter. C. difficile populations were monitored in all three vessels, and all other bacterial groups (total obligate anaerobes, total facultative anaerobes, lactose fermenting Enterobacteriaceae, Enterococci, total Clostridia, Lactobacilli, Bifidobacteria and B. fragilis group) were monitored in vessels 2 and 3 only. Vessel 3, which represents the distal colon, is of most physiological relevance in terms of propensity to induce CDI. C. difficile total viable counts and spores counts were monitored using viable counting and a differential alcohol shock viable count on selective agars. From day 14 onwards C. difficile cytotoxin was measured using a quantitative VERO cell cytotoxicity assay. Samples of 1 mL each were centrifuged at 16,000×g for 15 minutes, and the supernatants were removed. Six 1:10 serial dilutions (to 10⁻⁶) of culture supernatants from the gut model were prepared. Twenty microliters of the appropriate dilution was added to VERO cell monolayers and a further 20 μL of C. sordellii antitoxin (diluted 1:10 in sterile distilled water) was placed in the corresponding antitoxin row. Monolayers were examined after 24 and 48 hours of incubation in 5% CO₂, with a positive result indicated by the presence of cell rounding with concurrent neutralization of the effect by C. sordellii antitoxin. Cytotoxin titers (relative units, RU) were an arbitrary log₁₀ scale, and the cytotoxin titer is reported in the highest dilution with >70% cell rounding, i.e. 10⁰=1RU, 10⁻¹=2RU, 10⁻²=3RU. Samples were taken daily from day 21 onwards to determine antimicrobial concentrations in the gut model vessels by a bioassay. Concentrations of moxifloxacin were determined using Isosensitest agar with Escherichia coli as the indicator organism. Concentrations of omadacyline were determined using Wilkins Chalgren agar with Kocuria rhizophila as the indicator organism.

### Results

### Omadacycline Exposed Model

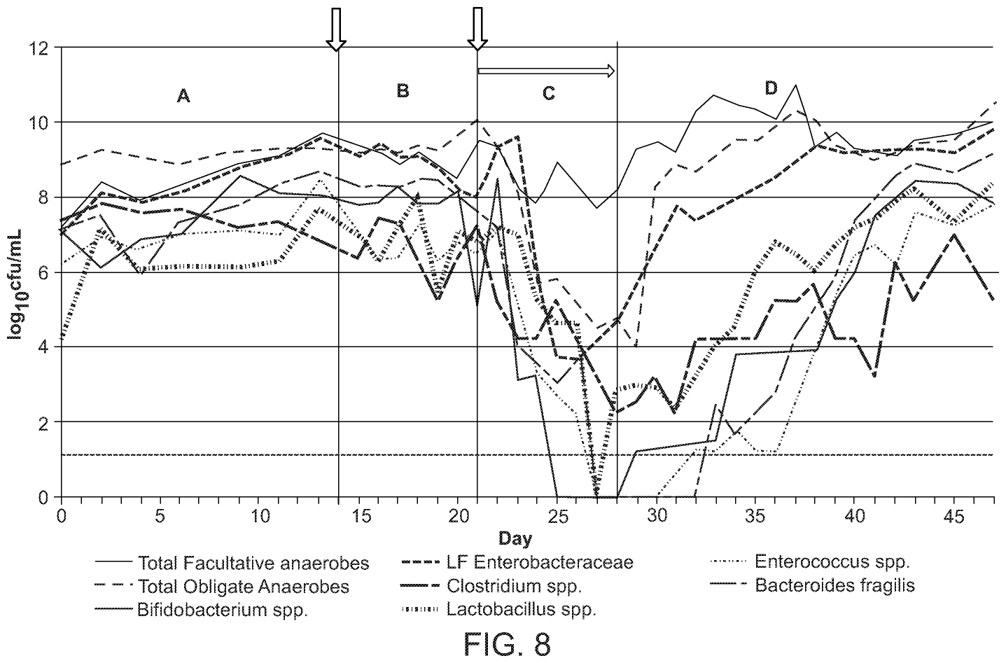

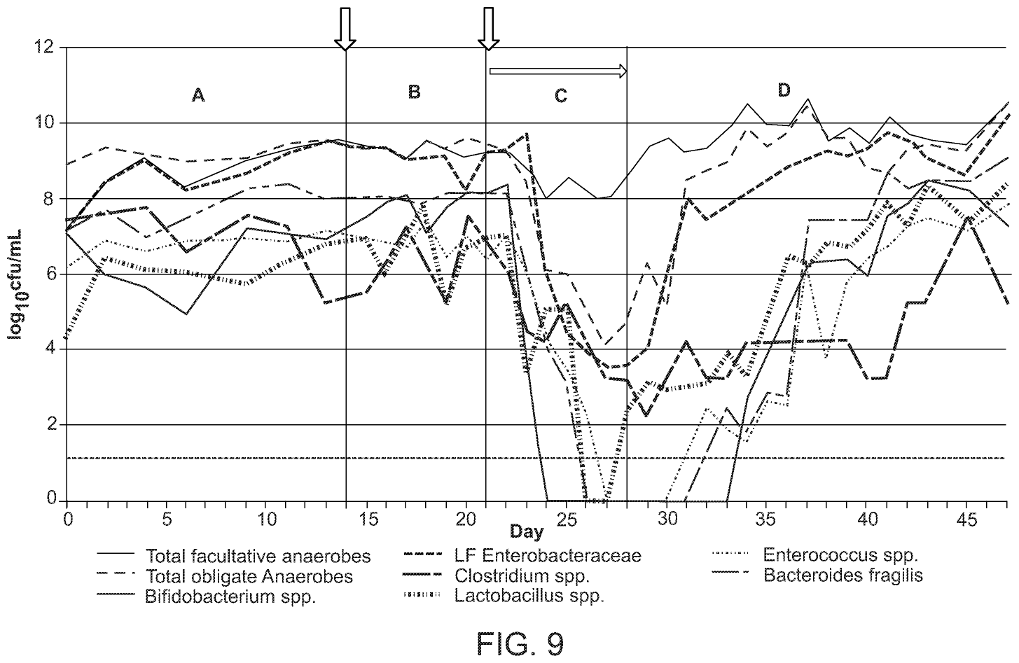

Changes in gut microflora populations were similar in vessels 2 and 3 (FIGS. 8 and 9). Omadacycline instillation caused marked declines in Bifidobacteria (˜8 log₁₀ cfu/mL), B. fragilis group (˜8 log₁₀ cfu/mL), Lactobacilli (˜6 log₁₀ cfu/mL) and Enterococcus spp. populations (˜6 log₁₀ cfu/mL), which all fell below the limit of detection. Decreases in Clostridia (˜5 log₁₀ cfu/mL), and lactose fermenting Enterobacteriaceae (˜5 log₁₀ cfu/mL) were also observed. Enterobacteriaceae populations increased during omadacycline exposure, particularly in vessel 2. These observations corresponded to an overall decline in total anaerobe populations of ˜5 log₁₀ cfu/mL. Total facultative anaerobes, however, remained fairly stable throughout. All populations recovered following the end of omadacyline dosing, and had returned to pre-antibiotic exposure levels by the end of the experiment.

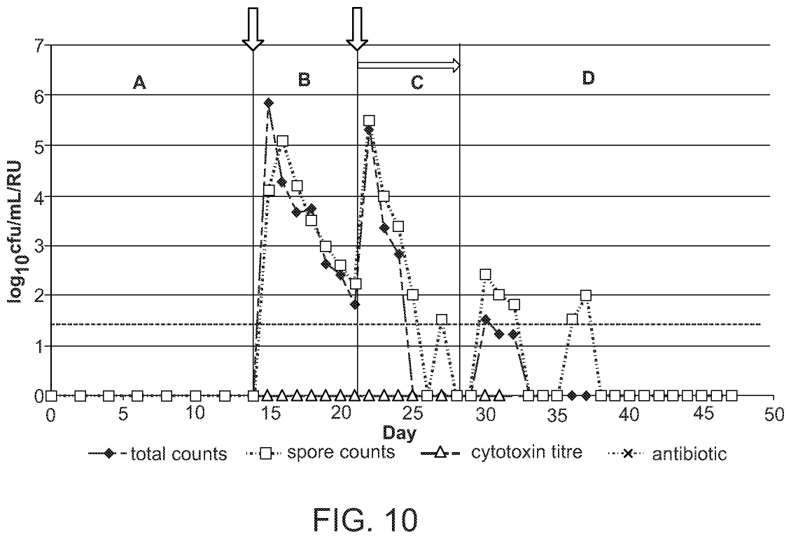

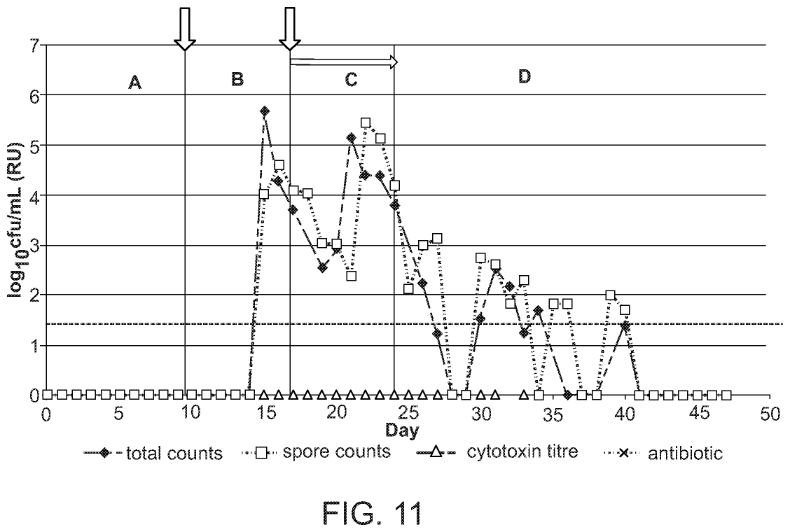

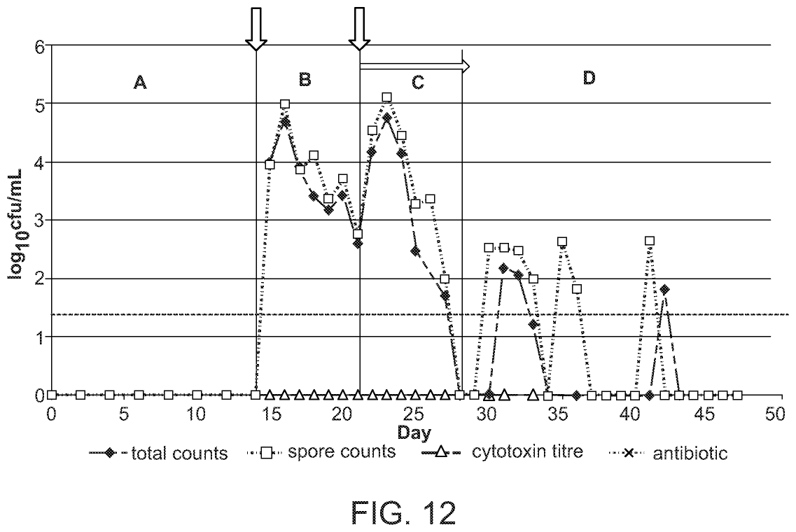

Despite extensive disruption of gut microflora population, omadacyline exposure did not lead to any signs of simulated CDI. C. difficile total viable counts (TVCs) remained roughly equal to spore counts throughout the experiment in all three vessels, indicating that all C. difficile remained as spores. There was no vegetative cell proliferation observed. No toxin was detected throughout this gut model experiment in any vessels (FIGS. 10, 11 and 12).

### Moxifloxacin Exposed Model

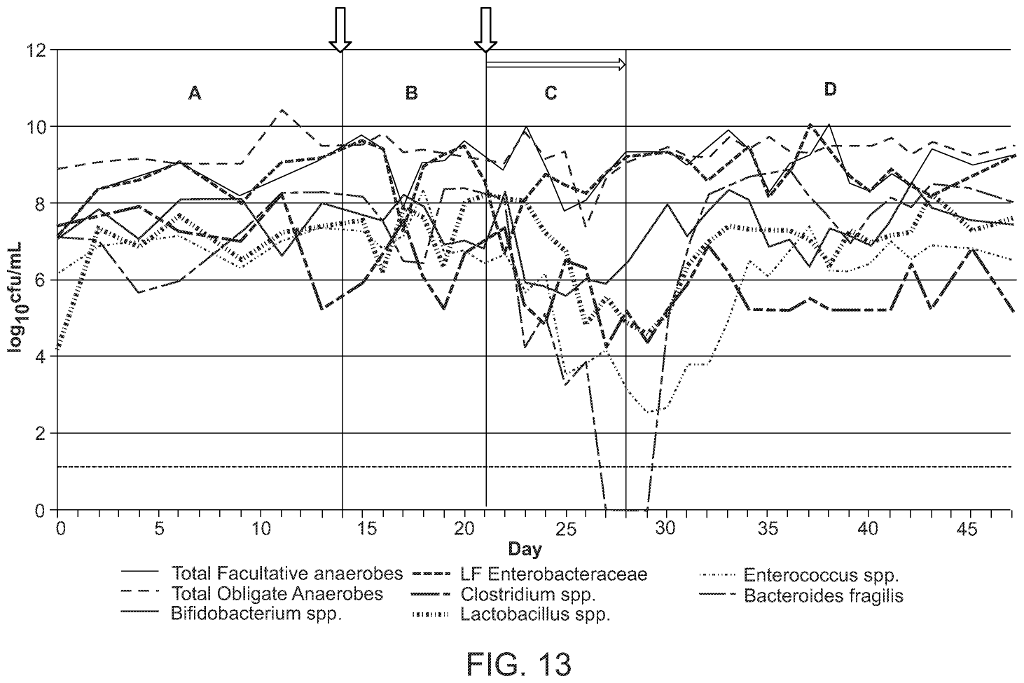

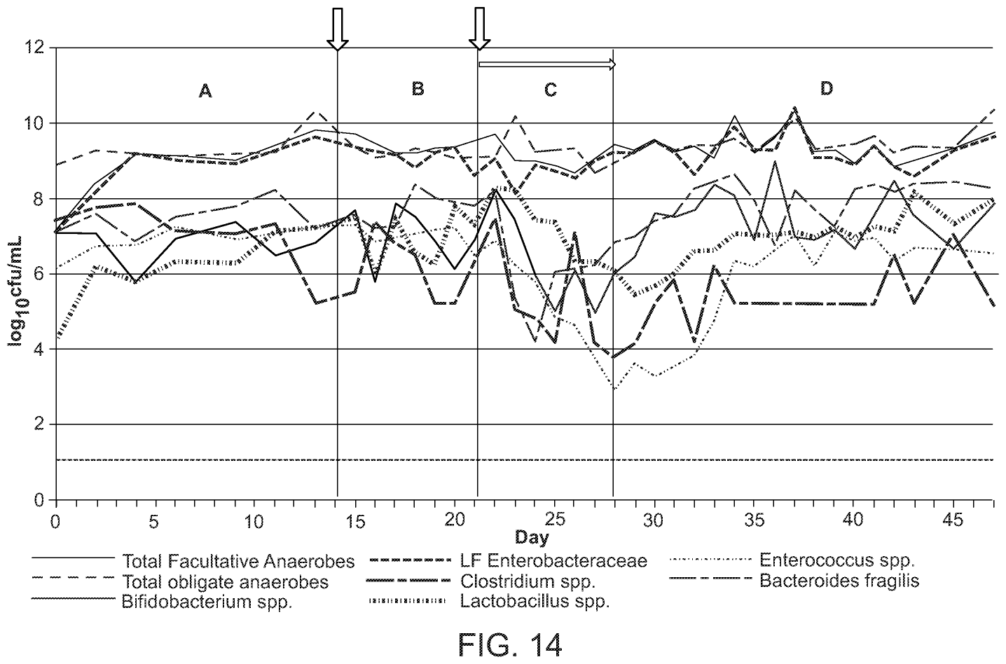

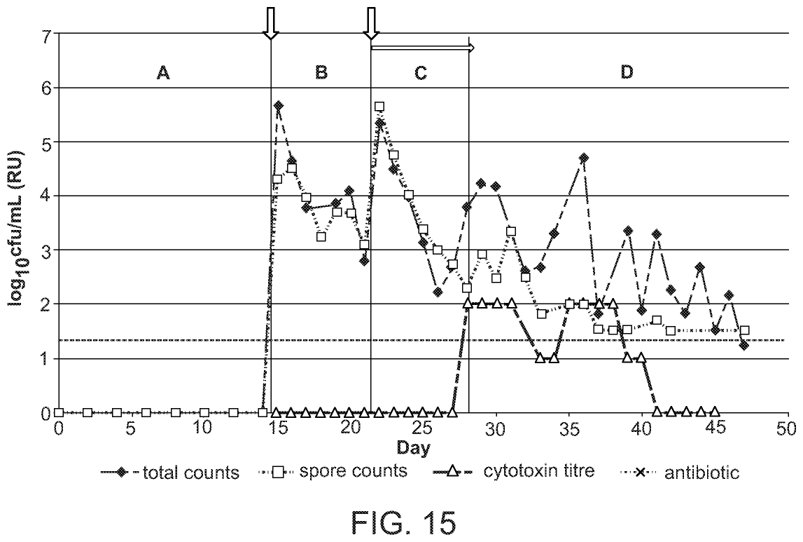

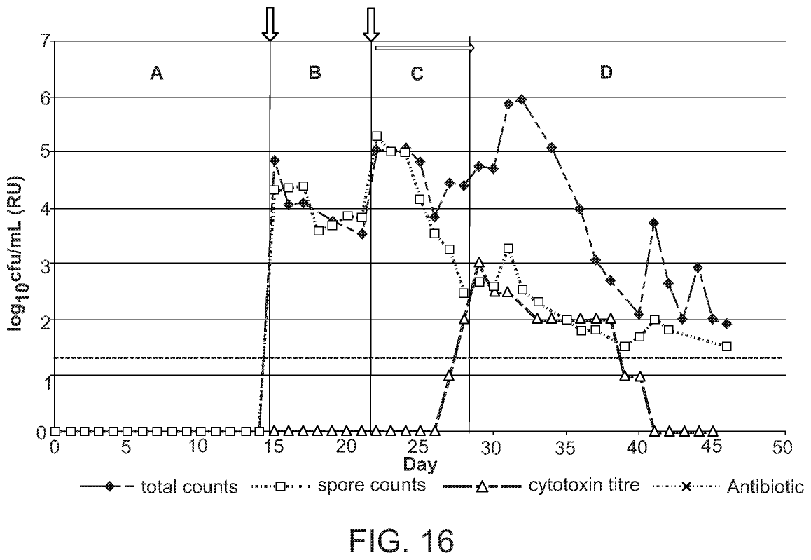

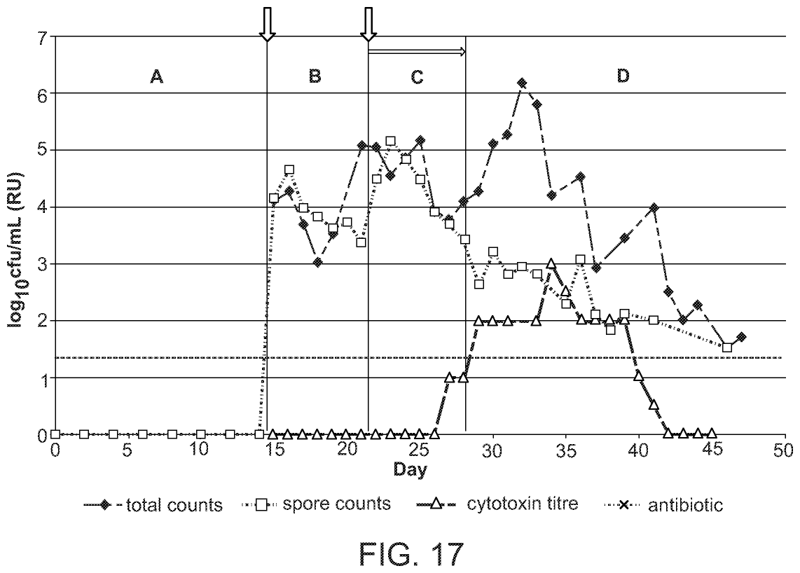

Changes in gut microbiota populations were similar in vessels 2 and 3 (FIGS. 13 and 14). Moxifloxacin instillation caused marked declines in B. fragilis group populations (˜8 log₁₀ cfu/mL in vessel 2 and ˜4 log₁₀ cfu/mL in vessel 3); Enterococci populations (˜4 log₁₀ cfu/mL in both vessel 2 and vessel 3); and Lactobacilli populations (˜3 log₁₀ cfu/mL in both vessel 2 and vessel 3). All populations returned to pre-antibiotic levels by ˜1 week following the end of antibiotic exposure.

In all three vessels, C. difficile remained as spores during the internal control period (B), but during moxifloxacin instillation, an increase in the total viable counts compared with spore counts was observed, indicating spore germination and vegetative cell proliferation. Total viable counts peaked at ˜4.5 log₁₀ cfu/mL in vessel 1, and ˜6 log₁₀ cfu/mL in vessel 2 and vessel 3. The increase in total viable counts was concomitant with the detection of C. difficile cytotoxin, which reached a peak titer of 2 relative units in vessel 1, and 3 relative units in vessel 2 and vessel 3. Both total viable counts and toxin titers decreased towards the end of the experiment, with toxin undetectable in all vessels by day 42.

### Discussion

Moxifloxacin instillation induced simulated CDI in the gut model in this study, with toxin detected in all three vessels. This is consistent with previous data demonstrating that moxifloxacin instillation causes substantial gut microflora disruption, and induces C. difficile spore germination, proliferation and toxin production (Saxton K et al., Antimicrob. Agents Chemother.; 53: 412-420, 2009). Moxifloxacin instillation had a marked effect on many components of the gut microbiota, including Bacteroides spp. (6 log₁₀ cfu/mL decline), lactose fermenting Enterobacteriaceae (6 log₁₀ cfu/mL decline), and Enterococci (4 log₁₀ cfu/mL decline) to below the limits of detection (Saxton et al., Antimicrob. Agents Chemother.; 53: 412-420, 2009), similar to the effects observed here. This disruption of gut microflora populations was followed by C. difficile spore germination, vegetative cell proliferation and detectable toxin.

Despite causing extensive disruption to the gut microflora, omadacycline exposure did not induce any signs of simulated CDI within the in vitro human gut model. This model has been shown to be clinically reflective. Antibiotics known to have a high propensity to induce CDI clinically have induced CDI in this model (e.g., clindamycin, cephalosporins, co-amoxyclav), whereas antibiotics considered as ‘low-risk’ for CDI clinically have not induced simulated CDI in the gut model (e.g., tigecycline and piperacillin-tazobactam). This study provides data indicating that omadacyline may be lower-risk for CDI induction, despite gut microflora effects disrupting ‘colonisation resistance. Notably, the lack of induction of CDI in the gut model by tigecycline and piperacillin-tazobactam was also despite marked gut microflora disruption (Baines et al., J. Antimicrob. Chemother. 55, 974-982, 2005; Baines et al., J. Antimicrob. Chemother., 58, 1062-1065, 2006). The high intrinsic activity of omadacycline, tigecycline and piperacillin-tazobactam against C. difficile presumably prevents its expansion even when a potential niche has been created by antibiotic exposure. Furthermore, the relatively rapid reconstitution of gut microflora populations after cessation of antibiotic will provide further protection against CDI.

When compared with the results of published and unpublished studies, which demonstrate that clinically relevant concentrations of moxifloxacin induce simulated CDI in the gut model, the data presented in Example 4 indicate that omadacycline is less likely to induce CDI than moxifloxacin and other fluoroquinolones.

Comparison with Example 3

The effects of omadacycline on anaerobic gut microbiota populations in Example 4 are similar to the effects observed in Example 3, with all measured anaerobic populations affected. The main difference between the data presented in Example 3 and Example 4 was observed in facultative anaerobic populations, for which a greater decline following omadacycline exposure was observed in Example 4 as compared to Example 3. As in Example 3, no signs of C. difficile germination, vegetative cell proliferation or toxin production were observed, indicating that omadacycline is less likely to induce CDI than other commonly used antibiotics. In Example 4, a comparator antibiotic, moxifloxacin, was also tested. The data in Example 4 indicate that omadacycline is less likely to induce CDI than moxifloxacin.

### EQUIVALENTS

Those skilled in the art will recognize, or be able to ascertain using no more than routine experimentation, many equivalents to the specific embodiments and methods described herein. Such equivalents are intended to be encompassed by the scope of the present invention. All patents, patent applications, and literature references cited herein are hereby expressly incorporated by reference.

1-7. (canceled)

8. A method of treating a bacterial infection without causing C. difficile infection in a subject who is at risk of developing a C. difficile infection, said method comprising administering to said subject an effective amount of a compound, or a salt thereof, wherein said compound is compound A′ having the following structural formula: 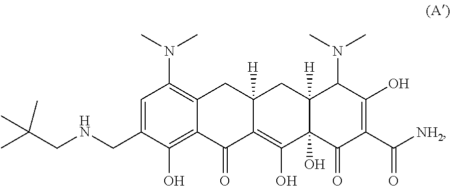 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]
such that said bacterial infection in said subject is treated without causing C. difficile infection.

9-10. (canceled)

11. The method of claim 8, further comprising, prior to administering, selecting a subject at risk of developing a C. difficile infection.

12. A method of treating a bacterial infection in a subject who is at risk of developing a C. difficile infection, said method comprising administering to said subject an effective amount of a compound, or a salt thereof, wherein said compound is compound A′ having the following structural formula: 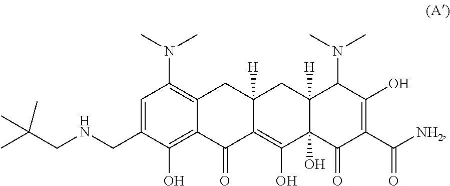 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]
such that said bacterial infection in said subject is treated.

13. The method of claim 12, said method comprising the steps of:
selecting a subject at risk of developing a C. difficile infection; and
administering to said subject an effective amount of a compound, wherein the compound is compound A′, or a salt thereof, having the following structural formula: 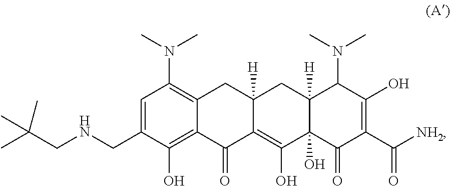 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]
such that said bacterial infection in said subject is treated.

14-19. (canceled)

20. The method of claim 8, wherein said compound is Compound A having the following structural formula:

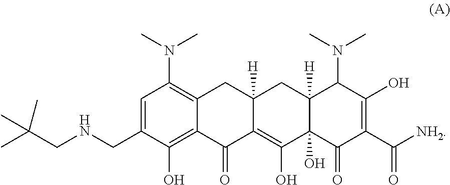 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

21. The method of claim 8, wherein the subject at risk of developing C. difficile infection is a subject selected from the group consisting of: a subject who was recently treated with one or more antibiotic; a subject who has had surgery of the gastrointestinal tract; a subject who has a disease of the colon; a subject who has a weakened immune system; a subject who is on chemotherapy; a subject who previously had a C. difficile infection; a subject who is 65 years or older; a subject who has a kidney disease; a subject who takes proton-pump inhibitors; and a subject who is living in an environment that predisposes said subject to developing a C. difficile infection.

22. The method of claim 21, wherein said antibiotic was a broad spectrum antibiotic.

23-24. (canceled)

25. The method of claim 21, wherein said disease of the colon is an inflammatory bowel disease or colorectal cancer.

26-32. (canceled)

33. The method of claim 21, wherein said environment comprises a hospital, a nursing home or an assisted living facility.

34-35. (canceled)

36. The method of claim 1, wherein said compound is administered orally or intravenously.

37-45. (canceled)

46. The method of claim 9, wherein the subject at risk of developing C. difficile infection is a subject selected from the group consisting of: a subject who was recently treated with one or more antibiotic; a subject who has had surgery of the gastrointestinal tract; a subject who has a disease of the colon; a subject who has a weakened immune system; a subject who is on chemotherapy; a subject who previously had a C. difficile infection; a subject who is 65 years or older; a subject who has a kidney disease; a subject who takes proton-pump inhibitors; and a subject who is living in an environment that predisposes said subject to developing a C. difficile infection.

47. The method of claim 13, wherein the subject at risk of developing C. difficile infection is a subject selected from the group consisting of: a subject who was recently treated with one or more antibiotic; a subject who has had surgery of the gastrointestinal tract; a subject who has a disease of the colon; a subject who has a weakened immune system; a subject who is on chemotherapy; a subject who previously had a C. difficile infection; a subject who is 65 years or older; a subject who has a kidney disease; a subject who takes proton-pump inhibitors; and a subject who is living in an environment that predisposes said subject to developing a C. difficile infection.

48. The method of claim 12, wherein said compound is Compound A having the following structural formula: 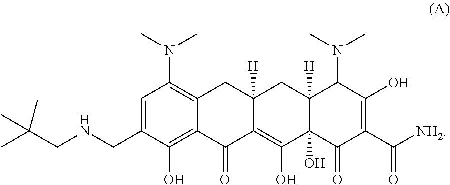 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]
